(19) United States
(12) Patent Application Publication
(10) Pub. No.: US 2012/0094271 A1
(43) Pub. Date: Apr. 19, 2012

(54) SELECTING FOR COOPERATIVELY INTERACTING MOLECULES

(75) Inventors: Jinglin Fu, Tempe, AZ (US); Neal W. Woodbury, Tempe, AZ (US); Stephen Albert Johnston, Tempe, AZ (US)

(73) Assignee: Arizona Board of Regents for and on behalf of Arizona State University, Scottsdale, AZ (US)

(21) Appl. No.: 13/061,941

(22) PCT Filed: Aug. 17, 2009
(86) PCT No.: PCT/US09/54078

§ 371 Date: Apr. 27, 2011

(30) Foreign Application Priority Data
Sep. 4, 2008 (US) PCT/US2009/054078

(51) Int. Cl. C12Q 1/70; C07K 14/00; C07H 21/00; C07H 21/02; C07H 3/06; C12N 7/00; C12N 1/20; C12Q 1/02; C07H 21/04; C22C 22/00; C22C 19/07; C22C 19/03; C22C 27/04; C22C 18/00; C22C 23/00; C22C 38/00; C22C 9/00; C22C 11/00; C22C 5/06; C22C 5/04; G01N 33/566; B82Y 15/00

(52) U.S. Cl. 435/5; 436/501; 530/350; 536/22.1; 536/23.1; 536/123.1; 435/235.1; 435/252.1; 435/29; 420/434; 420/435; 420/441; 420/429; 420/513; 420/402; 420/8; 420/469; 420/563; 420/501; 420/462; 977/773

(57) ABSTRACT

The present invention provides method of identifying molecules that cooperatively and positively interact with either a ligand or a target molecule of a ligand/target molecule pair, or molecules that interact with a ligand/target molecule complex.

### RELATED APPLICATIONS

This application claims priority to U.S. provisional application 61/094,250 filed on Sep. 4, 2008, which in incorporated herein in its entirety.

### BACKGROUND OF THE INVENTION

The field of high throughput analysis involving microarrays to study interactions between biomolecules has advanced quite steadily. Traditionally, the studies involving biomolecule interactions have focused on immobilizing biomolecules to maintain their activity while immobilized. There, however, remains a need to be able to identify new molecules that can interact with a ligand to improve the ligand's binding to its target molecule or to identify new molecules that can interact with a target molecule to improve the ligand's binding to the target molecule. There also remains a need to identify molecules that interact with the complex of a ligand and its target molecule. There is also a need to be able to identify new molecules that interact with the ligand only when the ligand is bound to its target molecule or interact with a target molecule only after it is bound to its ligand. The present invention fulfills this need. The present invention provides a platform with which to screen and identify these molecules as well as provides molecules identified using the methods described herein.

### SUMMARY OF THE INVENTION

The present invention provides methods of identifying a test molecule that can interact with a ligand of a ligand/target molecule pair to cooperatively interact with the ligand to improve its binding to its target.

The present invention also provides methods of identifying a test molecule that can interact with a target molecule of a ligand/target molecule pair to cooperatively interact with the target molecule to improve its binding to its ligand.

The invention also provides a method for identifying a test molecule that interacts with a ligand/target molecule complex.

The present invention further provides compositions con molecules identified by the methods of the invention.

### BRIEF DESCRIPTION OF THE FIGURES

FIG. 1 shows the comparison of the amount of β-Gal (beta-galactosidase) bound to each element of a peptide array (x-axis) compared with the amount of the enzyme/ligand complex bound to the same element of the peptide array (y-axis). In other words, this figure shows how the binding of β-Gal to each peptide in the array is affected by having prebound a specific peptide ligand to it. Primary peptide ligand: RVFKRYKRWLHVSRYYFGSC. Incubation conditions: 3 nM β-Gal was preincubated with 12 μM peptide ligand for half an hour in solution, then the enzyme-peptide mixture was incubated with peptide arrays for two hours; 3 nM β-Gal alone was incubated with peptide arrays for two hours as control experiments. (A) shows the comparison of median-normalized intensity values of the binding of β-Gal (X axis) versus the binding of peptide-β-Gal complex (Y axis). (B) shows the comparison of raw data intensity values of the binding of β-Gal (X axis) versus the binding of peptide-β-Gal complex (Y axis). The region marked with an ‘A’ in FIG. 1 represents the peptides that show a much stronger binding signal with the peptide-enzyme complex than enzyme alone.

FIG. 2 shows the selected peptides in FIG. 1 that only bind to the peptide-β-Gal complex. The left side is the binding intensity for enzyme alone and the right side is the binding intensity for the enzyme/primary ligand complex. (A) shows the median-normalized intensity values. (B) uses raw fluorescence data. The binding enhancement can be as high as ˜18 fold for normalization data and ˜30 fold for the raw data.

FIG. 3 shows the distribution in the isoelectric point (the pI) of the test molecules used in FIG. 2. Note that there is a distribution of pI values between 3 and 11 with most of the test peptides selected having pI values near neutral.

FIG. 4 shows the binding enhancement of seven selected secondary peptide binders to β-Gal/primary ligand complex.

FIG. 5 provides a diagram of five mechanisms for coo between a Test molecule (T) on the surface, a target molecule (P) and a Ligand molecule (L).

### DETAILED DESCRIPTION OF THE INVENTION

The present invention provides a method for identifying a test molecule that cooperatively and positively interacts with a ligand of a ligand/target molecule pair (e.g. the test molecule and the ligand cooperate together to provide an improved binding/interaction of the ligand to its target molecule as compared to the binding of the ligand to is target molecule alone). In other words: 1) the ligand may affect the binding of the test molecule to the target molecule; 2) the test molecule may affect the binding of the ligand to the target molecule; or 3) the ligand and the test molecule interact together so that together they bind to the target molecule better than they would bind alone.

In another embodiment, the method provides a method for identifying a test molecule that cooperatively and positively interacts with a target molecule of a ligand/target molecule pair (e.g. the test molecule and the target molecule cooperate together to provide an improved binding/interaction of the target molecule to its ligand as compared to the binding of the target molecule to its ligand alone). In other words: 1) the target molecule may affect the binding of the test molecule to the ligand; 2) the test molecule may affect the binding of the target molecule to the ligand; or 3) the test molecule and the target molecule interact together so that together they bind to the ligand better than they would bind than alone.

The present invention further provides a method for identifying a test molecule that cooperatively and positively interacts with a ligand/target molecule complex. For example, the ligand and the target molecule bind to form a complex and their interaction forms a new binding site made by the physical junction of both the ligand and the test molecule. The test molecule binds this new binding site. Alternatively, the test molecule binds the ligand portion of the ligand/target molecule complex or in another embodiment, the test molecule binds the target molecule portion of the complex. In certain embodiments, the test molecule binds the complex better than binding the ligand or the target alone, and in other embodiments, the test molecule does not bind the ligand or the target alone and only binds when molecule are complexed together.

The test molecule, ligand and/or target molecule may be any entity such as, but not limited to, a biomolecule, including but not limited to, a peptide, peptoid, protein, nucleic acid, DNA/RNA aptamers, protein PNA molecule, oligosacharride, heteropolymer, cell membrane, virus, phage, cellular organelle, bacterium or eukaryotic cell, etc. The test molecule, ligand and/or target molecule may also be any small molecule, a metal ion such as but not limited to Manganese (Mn), Cobalt (Co), Nickel (Ni), Molybdenum (Mo), Zinc (Zn), Magnesium (Mg), Iron (Fe), Copper (Cu), Lead (Pd), Ruthenium (Ru) or Silver (Ag), a nanoparticle or a nonbiological heteropolymer or macromolecule, for example.

Any known ligand-target molecule interaction can be used in the methods of the present invention. The ligand is any entity as discussed above that has the ability to react, bind or associate with a target molecule. The target molecule may be any entity having the ability to react, bind or associate with a ligand.

Binding as used herein refers to binding, associating, or interacting with each other.

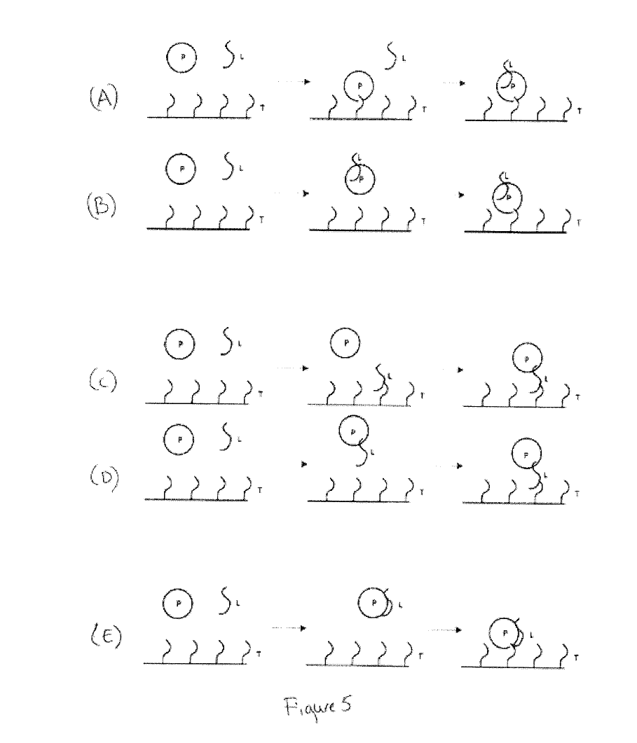

In certain embodiments of the invention, the methods involve identifying a molecule from a pool of test molecules that can cooperatively and positively interact with a ligand of a ligand/target molecule pair to better bind its target molecule. For example, a test molecule may interact with the ligand so that the ligand-test molecule pair better binds the target molecule (stronger or longer binding, for example) as compared to the binding of the ligand or the test molecule to the target molecule alone. The test molecule may alter the ligand in such a way to change its binding constant to allow it to better bind its target. See FIG. 5C. In another embodiment, the ligand and its target can bind to form a complex, which then binds the test molecule through a site on the ligand. See FIG. 5D.

The method comprises exposing at least one test molecule from a pool of test molecules to the target molecule and identifying a test molecule that binds to the target molecule. The degree of binding of the test molecule to the target molecule is determined. The ligand and its target molecule are then exposed to the identified test molecule and the degree of binding of the test molecule to the target molecule in the presence of the ligand is determined. In certain embodiments, i [symbol] above, the ligand alone may be exposed to the test molecule and the degree of binding is determined. The degree of binding from these steps are compared to allow the identification of a test molecule that interacts with the ligand to provide a better binding of the ligand to its target molecule.

In other embodiments of the invention the method involves identifying a molecule from a pool of test molecules that can cooperatively and positively interact with a target molecule of a ligand/target molecule pair to better bind to its ligand. For example, a test molecule may interact with the target molecule so that the test molecule-target molecule pair better binds the ligand as compared to the binding of the test molecule or the target molecule to the ligand alone. The test molecule may alter the target molecule in such a way to change its binding constant to allow it to better bind its ligand. See FIG. 5A. In another embodiment, the ligand and its target can bind to form a complex, which then binds the test molecule through a site on the target molecule. See FIG. 5B.

The method comprises exposing a pool of test molecules to a ligand and identifying a test molecule that binds to the ligand. The degree of binding of the test molecule to the ligand is determined. The ligand and its target molecule are exposed to the identified test molecule, and the degree of binding of the test molecule to the ligand in the presence of the target molecule is determined. In certain embodiments, in addition to the steps above, the target molecule alone, may be exposed to the test molecule and the degree of binding is determined. The degree of binding from these steps are compared to allow the identification of a test molecule that interacts with the target molecule to provide a better binding of the target molecule to its ligand. Similarly, as discussed above, the ligand and the target molecule may be allowed to bind, interact or associate before being exposed to the test molecule.

The present invention also provides a method of identifying a test molecule that binds to a ligand/target molecule complex where the test molecule does not bind the ligand or test molecule independently. This method comprises exposing at least one test molecule to the ligand/target molecule complex and identifying the at least one test molecule that binds to the ligand/target molecule complex. The degree of binding of the least one test molecule to the ligand/target molecule complex is determined. The test molecule is then exposed to the ligand alo target molecule alone and the degree of binding is determined. The degree of binding from the steps above are compared to identify the at least one test molecule that binds to the ligand/target molecule complex and does not bind to the either the ligand alone or the target molecule alone.

The present invention also provides a method for identifying a test molecule that binds to a ligand/target molecule complex. For example, when the ligand and the target molecule bind to form a complex, they create a site in which the test molecule will bind. The test molecule may bind at the physical junction of both the ligand and the target molecule. See FIG. 5E. In this case, all three molecules are physically interacting with another in a direct fashion. Mechanistically, the order of binding does not matter. For example, the target molecule can bind the ligand and this complex then binds the test molecule. In this situation, the method would comprise allowing the ligand and the target molecule to form a complex before exposing it to the test molecule. In another scenario, the test molecule can bind the ligand and then this complex can bind the target. In another scenario, the test molecule can bind the target molecule and then this complex can bind the ligand. Since the binding involves the interaction of all three entities, the degree of binding between the test molecule with the ligand/target molecule would be stronger than the binding of the test molecule to the ligand or target molecule alone.

In certain embodiments of the invention, the ligand and the target molecule may be first allowed to associate, bind or interact with each other before exposure to the test molecule or they may be each added independently to the test molecule.

In certain embodiments, the ligand and the test molecule may be linked together by a covalent linker to form a binding element comprised of the linker, the ligand and the test molecule.

The methods described above can identify test molecules that interact various ways with the ligand, the target molecule or the complex of the ligand and the target. There are several types of cooperative interactions that can take place. Thermodynamically, they are equivalent, but mechanistically they are different. For example, in certain embodiments, one can have the test molecule on the surface interact with a ligand molecule in solution and this complex can then bind the target molecule better than either one alone. In other embodiments, or molecule on the surface interact with the target molecule in solution and this complex can bind the ligand better than either the test molecule or the target alone. In other embodiments, one can have the ligand molecule bind the target and that complex can bind the test molecule better than either the ligand or target alone.

The actual binding interaction in each case above can involve the ligand and the test molecule binding to different, separate regions of the target, but interacting through some change in the target that happens when one or the other binds. Alternatively, the ligand molecule can bind at the test molecule and the target, without the test molecule ever actually contacting the target. In another scenario, all three molecules can bind together such that all three molecules are physically interacting with each other.

FIG. 5 provides a diagram of five exemplary mechanisms for cooperative binding between a test molecule (T) on the surface, a target molecule (P) and a ligand molecule (L). The top two mechanisms (A and B) involve binding of the ligand and the test molecule at different sites on the target. The binding of either the test molecule (T) or the ligand (L) effects the other in such a way as to change its binding constant. In the first case (A), the target (P) binds to the test molecule (T) and then the ligand (L) binds to the target. In the second case (B), the ligand (L) binds to the target (P) and then the complex binds to the test molecule (T). The second two mechanisms (C and D) involve binding of the target to the ligand (L) at a region of the ligand separate from where the ligand (L) binds the test molecule (P) on the surface. Binding of the test molecule (T) to the ligand (L) changes the binding affinity of the target (P) for the ligand (L) and binding of the target (P) to the ligand (L) changes the binding affinity of the test molecule (T) for the ligand. The binding can occur in two ways as shown. The ligand (L) can first bind to the test molecule (T) and then the target can bind to the ligand (C), or the target and the ligand (L) can bind and the complex then binds to the test molecule (T) through a site on the ligand (D). Finally, the last mechanism (E) demonstrates the case where the target and the ligand (L) bind and form a new site made by the physical junction of both entities and this new site binds to the test molecule (T). In this case all three molecules are physically interacting with one another in a direct fashion. Mechanistically, order does not matter. The target can bind the ligand as shown and this compl [symbol] molecule or the target can bind to the test molecule and then the ligand can bind to that complex (not shown) or the ligand can bind to the test molecule and that complex can bind the target (not shown). The difference between the last set of mechanisms and the first two sets is just that all three molecules in the final complex directly interact physically.

After a desired test molecule is identified by any of the methods of the present invention, the degree of cooperative binding can then be determined by more detailed binding studies using methods for evaluating cooperative binding known to those in the art.

The methods of the present invention can be carried out by methods known in the art that have the ability to test the binding of a target or target/ligand complex to a set of test molecules. For example, the set of test molecules is in an array affixed to a surface and the target molecule or the ligand are labeled, allowing one to determine which elements in the array bind to the target or target/ligand complex. Another example utilizes surface plasmon resonance (SPR). For example, the A-100 instrument from Biacore can be used to measure the binding of thousands of different molecules in a test set to one or more target molecules or target/ligand complexes affixed to a surface. In this case, it might be desirable to crosslink the ligand to the target molecule for such a test and then directly assay binding of the test molecule set, comparing, on the same SPR chip, target molecules with and without the ligand crosslinked to them.

In addition, there are a variety of methods known in the art that involve affixing each molecule in the test molecule set to an individual bead and then identifying those beads that bind the target and/or the target/ligand complex. It is also possible to use uncomplexed target that is unlabeled as a competitor molecule so that only the test molecules that bind the target/ligand complex are detected. It may be desirable to crosslink the ligand to the target molecule.

As another non-limiting example, it is also possible to attach the target molecule to beads in a chromatography column and put a mixture of test molecules over the column, later eluting only test molecules that bound to the column (with or without the ligand). These molecules may then be identified by spectroscopy.

The present invention provides a composition comprising a test molecule that binds a ligand of a ligand/target molecule pair wherein the test molecule only binds the ligand after the ligand binds its target molecule or wherein the test molecule binds the ligand more strongly after the ligand binds its target molecule.

The invention further provides a composition comprising a test molecule that binds a target of a ligand/target molecule pair wherein the test molecule only binds the target after the target binds the ligand or wherein the test molecule binds the target more strongly after the target binds the ligand.

The present invention also provides a composition comprising a test molecule that binds a ligand/target molecule complex only after the complex has formed and wherein the test molecule does not bind the ligand or the target molecule alone. In certain embodiments, the test molecule binds the ligand/target molecule complex at a physical junction of the ligand and the target molecule. In other embodiments, the test molecule binds the ligand portion after the ligand/target molecule has formed a complex. In other embodiments, the test molecule binds the target molecule after the ligand/target molecule has formed a complex.

The present invention also provides a composition comprising a test molecule that binds a ligand/target molecule complex more strongly than it binds either the target molecule or ligand alone.

The present invention also provides a molecule or compositions comprising a molecule identified by any of the methods of the present invention.

### EXAMPLES

### Example 1

### Identifying Two Peptides that Bind Cooperatively to Beta-Galactosidase

Beta-galactosidase (β-gal) (˜3 nM, Alexa647 labeled, the target) was preincubated with a solution containing ˜12 uM of the peptide “RVFKRYKRWLHVSRYYFGSC” (the ligand). This peptide binds strongly to the β-gal enzyme at this concentration and inhibits its activity. In fact, at this concentration, 95% of β-gal activity is inhibited by this peptide. The complex between β-gal and the peptide complex was incubated with a pe [symbol] 10,000 random sequence peptides (the “test molecules”) bound to the surface of a glass slide in an ordered fashion. The incubation took place for two hours and the binding pattern of this was compared to the binding pattern using only the labeled enzyme and no ligand.

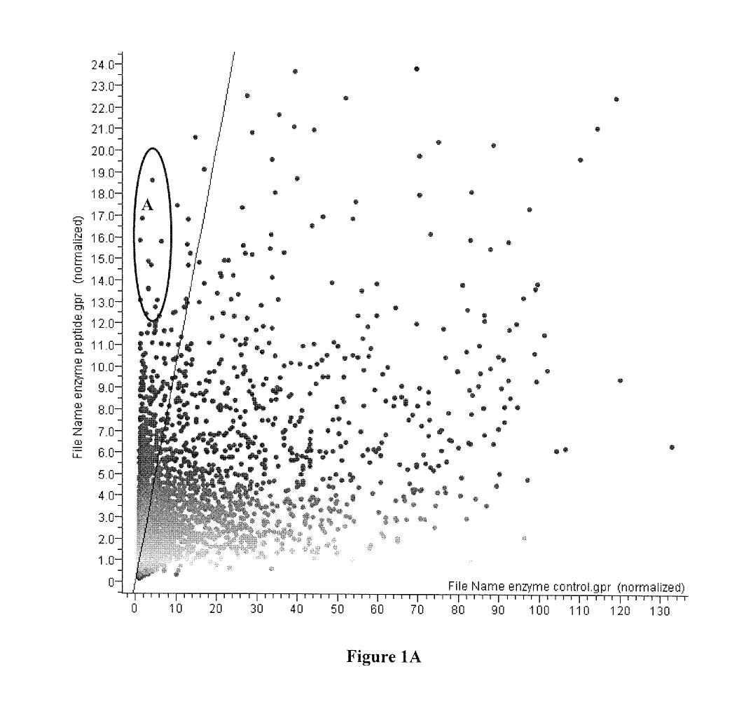

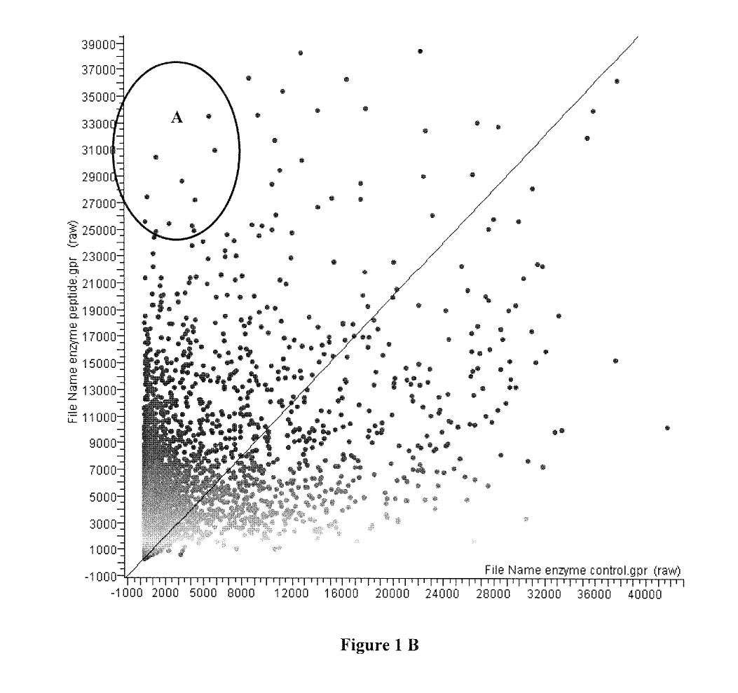

FIG. 1A compares the amount of binding of β-Gal itself to each element of the peptide array with the amount of binding of the enzyme/primary ligand complex. In particular, the X axis is the binding to the enzyme alone and the Y axis is the binding to the enzyme/ligand complex. The data in FIG. 1A was a median-level normalized intensity. All the raw signals were normalized to the 50^{th} percentile of the measurements under a particular set of conditions by first determining the binding at each position under a particular set of conditions, then determining the 50^{th} percentile binding intensity of all the test molecules on the array then finally dividing the result for each by the 50^{th} percentile binding intensity. The region marked with an ‘A’ in FIG. 1A represents the peptides that show a much stronger binding signal when the peptide-enzyme complex is bound to the array than when just the enzyme is bound alone. These peptides are good candidates for binding cooperatively (positively cooperatively in this case) with the primary ligand to the target.

FIG. 1B shows the raw data (fluorescence intensities due to target binding), plotted in the same way as FIG. 1A. It shows clearly that on average the binding between the primary ligand-enzyme complex and the test peptides on the array is greater than what is seen for the labeled β-Gal alone. The region marked with an ‘A’ in FIG. 1B represents the peptides that show a stronger binding signal with the peptide-enzyme complex than enzyme alone.

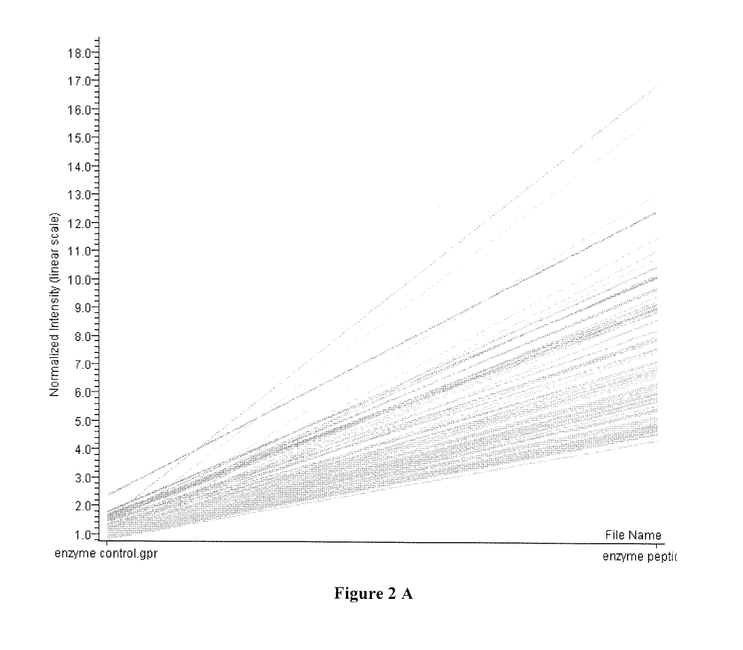

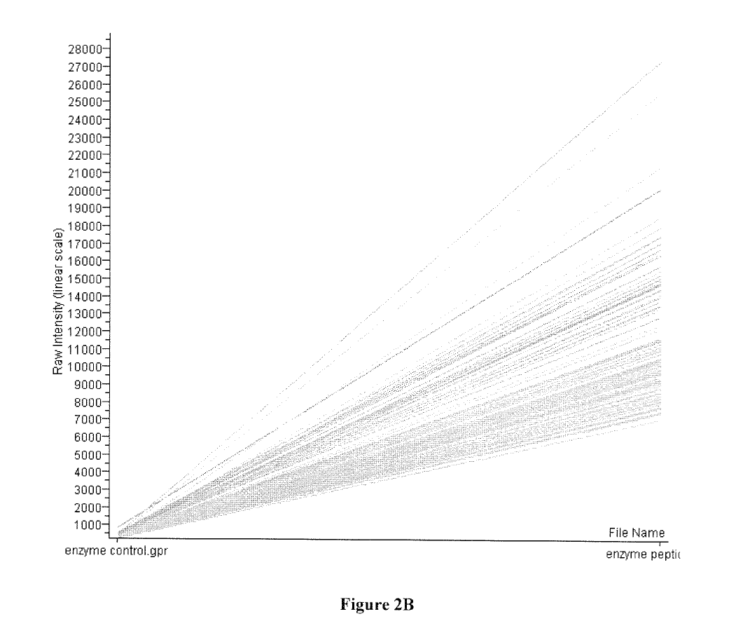

FIG. 2 shows the selected peptides in FIG. 1 that only bind to the peptide-β-gal complex. The left side is the binding intensity for enzyme alone and the right side is the binding intensity for the enzyme/primary ligand complex. (A) shows the median-normalized intensity values. (B) uses raw fluorescence data. The binding enhancement can be as high as ˜15 fold for normalization data and ˜76 fold for the raw data.

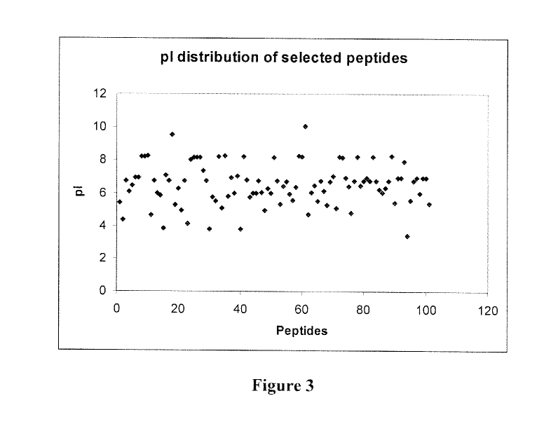

The distribution in the isoelectric point (the pI) of the selected peptides in FIG. 2 is shown in FIG. 3. Note that there is a distribution of pI values between 3 and 11 with most of the test peptides selected having pI values r [symbol] very different form the pI (10.6) of the ligand peptide

“RVFKRYKRWLHVSRYYFGSC.”

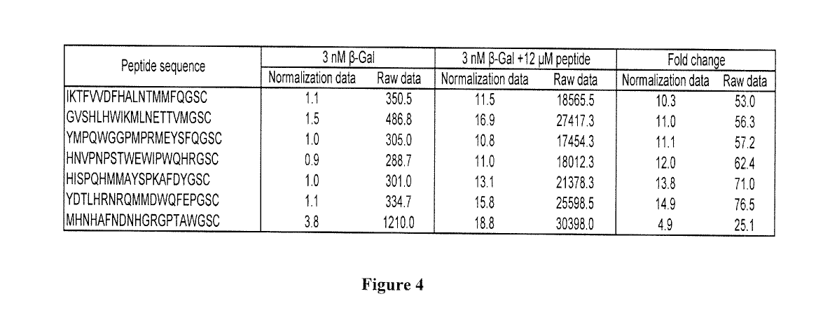

FIG. 4 shows the binding enhancement of seven selected secondary peptide binders to β-Gal/primary ligand complex.

Overall, this example shows that it is possible to find a test peptide on the surface that binds much more strongly to the ligand-enzyme complex than it does to the enzyme alone.

1. A method for identifying a test molecule that interacts with a ligand of a ligand/target molecule pair to identify the test molecule that cooperates with the ligand to improve binding of the ligand or binding of the test molecule to the target molecule, the method comprising:
a) exposing at least one test molecule to the target molecule and identifying the at least one test molecule that binds to the target molecule;
b) determining the degree of binding of the least one test molecule to the target molecule;
c) exposing the ligand and its target molecule to the identified test molecule from step (a) and determining the degree of binding of the least one test molecule to the target molecule in the presence of the ligand; and
d) comparing the degree of binding from steps (b) and (c) and identifying the at least one test molecule that binds better to the target molecule in the presence of the ligand to identify the test molecule that cooperates with the ligand to improve binding of the ligand or binding of the test molecule to the target molecule.

2. A method for identifying a test molecule that interacts with a ligand of a ligand/target molecule pair to identify the test molecule that cooperates with the ligand to improve binding of the ligand or binding of the test molecule to the target molecule, the method comprising:
a) exposing at least one test molecule to the target molecule and identifying the at least one test molecule that binds to the target molecule;
b) determining the degree of binding of the least one test molecule to the target molecule;
c) exposing the ligand and its target molecule to the identified test molecule from step (a) and determining the degree of binding of the least one test molecule to the target molecule in the presence of the ligand;
d) exposing the ligand to the at least one test molecule and determining the degree of binding of the at least one test molecule to the ligand; and
e) comparing the degree of binding from steps (b), (c) and (d) and identifying the at least one test molecule that binds better to the target molecule in the presence of the ligand to identify the test molecule that cooperates with the ligand to improve binding of the ligand or binding of the test molecule to the target molecule.

3. A method for identifying a test molecule that interacts with a target molecule of a ligand/target molecule pair to identify the test molecule that cooperates with the target molecule to improve binding of the target molecule or binding of the test molecule to the ligand, the method comprising:
a) exposing at least one test molecule to the ligand and identifying the at least one test molecule that binds to the ligand;
b) determining the degree of binding of the least one test molecule to the ligand;
c) exposing the ligand and its target molecule to the identified test molecule from step (a) and determining the degree of binding of the least one test molecule to the ligand in the presence of the target molecule; and
d) comparing the degree of binding from steps (b) and (c) and identifying the at least one test molecule that that interacts with a target molecule of a ligand/target molecule pair to identify the test molecule that cooperates with the target molecule to improve binding of the target molecule or binding of the test molecule to the ligand.

4. A method for identifying a test molecule that interacts with a target molecule of a ligand/target molecule pair to identify the test molecule that cooperates with the target molecule to improve binding of the target molecule or binding of the test molecule to the ligand, the method comprising,
a) exposing at least one test molecule to the ligand and identifying the at least one test molecule that binds to the ligand;
b) determining the degree of binding of the least one test molecule to the ligand;
c) exposing the ligand and its target molecule to the identified test molecule from step (a) and determining the degree of binding of the least one test molecule to the ligand in the presence of the target molecule;
d) exposing the target molecule to the at least one test molecule and determining the degree of binding of the at least one test molecule to the target molecule; and
e) comparing the degree of binding from steps (b), (c) and (d) and identifying the at least one test molecule that that interacts with a target molecule of a ligand/target molecule pair to identify the test molecule that cooperates with the target molecule to improve binding of the target molecule or binding of the test molecule to the ligand.

5. A method for identifying a test molecule that binds to ligand/target molecule complex wherein the test molecule does not bind to the ligand or the target molecule alone, the method comprising:
a) exposing at least one test molecule to the ligand/target molecule complex and identifying the at least one test molecule that binds to the ligand/target molecule complex;
b) determining the degree of binding of the least one test molecule to the ligand/target molecule complex;
c) exposing the test molecule to the ligand alone and determining the degree of binding of the least one test molecule to the ligand;
d) exposing the test molecule to the target molecule alone and determining the degree of binding of the at least one test molecule to the target molecule; and
e) comparing the degree of binding from steps (b), (c) and (d) and identifying the at least one test molecule that binds to the ligand/target molecule complex and does not bind to the ligand alone and the target molecule alone.

6. A method for identifying a test molecule that binds to a ligand/target molecule complex wherein the test molecule binds a site created by the physical junction of both the ligand and the target molecule, the method comprising:
a) exposing at least one test molecule to the ligand/target molecule complex and identifying the at least one test molecule that binds to the ligand/target molecule complex;
b) determining the degree of binding of the least one test molecule to the ligand/target molecule complex;
c) exposing the test molecule to the ligand alone and determining the degree of binding of the least one test molecule to the ligand;
d) exposing the test molecule to the target molecule alone and determining the degree of binding of the at least one test molecule to the target molecule; and
e) comparing the degree of binding from steps (b), (c) and (d) and identifying the at least one test molecule that binds to the ligand/target molecule complex better than binding to the ligand alone and the target molecule alone.

7. The method of claims 1 through 4 wherein the ligand and its target molecule are allowed to interact to form a complex before step (c).

8. The method of claims 1 through 4 wherein the ligand and the at least one test molecule are linked together by a covalent linker to form a binding element comprised of the linker and the ligand and the at least test one molecule.

9. A composition comprising a test molecule that binds a ligand of a ligand/target molecule pair wherein the test molecule only binds the ligand after the ligand binds the target molecule or wherein the test molecule binds the ligand more strongly after the ligand binds the target molecule.

10. A composition comprising a test molecule that binds a target of a ligand/target molecule pair wherein the test molecule only binds the target after the target binds the ligand or wherein the test molecule binds the target more strongly after the target binds the ligand.

11. A composition comprising a test molecule that binds a ligand/target molecule complex only after the complex has formed and wherein the test molecule does not bind the ligand or the target molecule alone.

12. A composition comprising a test molecule that binds a ligand/target molecule complex more strongly than the test molecule binds to the individual ligand or target molecule.

13. The composition of claim 11 wherein the test molecule binds the ligand/target molecule complex at a physical junction of the ligand and target molecule.

14. The composition of claim 11 wherein the test molecule binds the ligand portion of the ligand/target molecule complex.

15. The composition of claim 11 wherein the test molecule binds the target molecule portion of the ligand/target molecule complex.

16. The test molecule identified by the method of claim 1 or 2.

17. The test molecule identified by the method of claim 3 or 4.

18. The test molecule identified by the method of claim 5.

19. The test molecule identified by the method of claim 6.

20. The test molecule of claim 16 wherein the ligand binds its target molecule at a separate site from the test molecule and the binding interaction between the ligand and the test molecule is allosteric.

21. The test molecule of claim 16 wherein the ligand alters the surface of its target molecule and the test molecule binds directly to the surface of the target molecule.

22. The test molecule of claim 17 wherein the structure of the ligand is altered upon binding to its target molecule and the test molecule binds to the altered ligand and wherein the test molecule does not directly bind to the target molecule.
